(19) United States
(12) Patent Application Publication
(10) Pub. No.: US 2006/0100511 A1
(43) Pub. Date: May 11, 2006

(54) Tube

(76) Inventor: Morten Eriksen, Nydalen (NO)

Correspondence Address: GE HEALTHCARE, INC., IP DEPARTMENT, 101 CARNEGIE CENTER, PRINCETON, NJ, 08540-6231, US

(21) Appl. No.: 10/530,745

(22) PCT Filed: Oct. 8, 2003
(86) PCT No.: PCT/NO03/00336

§ 371 Date: Aug. 22, 2005

(30) Foreign Application Priority Data
Oct. 9, 2002 (NO) 20024883

(51) Int. Cl. A61B 6/00

(52) U.S. Cl. 600/431; 604/264; 264/165

(57) ABSTRACT

The invention relates to a device and method of using the device for administration of a homogeneous preparation to a patient. More particularly the invention relates to a tube for use in controlled and substantially steady state administration of a segregating particulate dispersion by infusion. The tube of the invention has a non-circular cross-section and is twisted along its centerline.

### FIELD OF THE INVENTION

This invention relates to a device and method of using the device for administration of a homogeneous preparation to a patient. More particularly the invention relates to a tube for use in controlled and substantially steady state administration of a segregating particulate dispersion by intravenous infusion.

### DESCRIPTION OF THE RELATED ART

In the field of ultrasonography it is well known that gas-containing contrast agents are particularly efficient backscatterers of ultrasound by virtue of the low density and ease of compressibility of the microbubbles. Such microbubble dispersions, if appropriately stabilized, may permit highly effective ultrasound visualization of, for example, the vascular system and tissue microvasculature, often at advantageously low doses of the contrast agent.

Continuous infusion of ultrasound contrast agents, for example over a period in the range from one minute to one hour, is of potential interest in that it may permit administration of the contrast agent at a rate which minimizes diagnostic artifacts such as shadowing and may lengthen the useful time window for imaging beyond the relatively short duration of the backscatter signal peak resulting from passage of a contrast agent bolus.

A problem with the continuous infusion of gas-containing ultrasound contrast agents arises from the tendency of gas-containing components such as microbubbles to float, since this may lead to inhomogeneities forming within receptacles and other delivery equipment which may be used in administering the contrast agent. This may, for example, lead to an increase in microbubble concentration in the upper part of such receptacles and/or to changes in microbubble size distribution occurring at various points within the receptacles as larger microbubbles float more rapidly than smaller microbubbles.

There is hence an ongoing need for apparatus that permits the continuous infusion of gas-containing ultrasound contrast agents or other gravity segregating particulate dispersions while maintaining substantial homogeneity of the contrast agent or other dispersion.

A typical apparatus for administration of an ultrasound contrast agent to a patient would comprise a delivery receptacle, such as a syringe coupled to a syringe pump, and a tube connecting the delivery receptacle to a needle for injection to a patient's vein.

Different methods and devices for maintaining a homogeneous fluid preparation during administration have been described earlier. WO 00/53242 describes devices, systems and methods for dispensing a multi-component medium subjecting the medium to different agitation mechanisms. Rotation of a storage volume, such as a cartridge, is one of the described ways of achieving agitation of a multi-component medium.

WO 00/71189 describes contrast media resuspension devices and methods wherein a volume of a sedimenting agent is divided into a network of sub-volumes by incorporating tubes, cells, sponges or grooves in the volume. It is further described that the internal geometry of such network may be non-circulatory.

However, during infusion of ultrasound contrast agents flotation of microbubbles in the tube that connects the injection system (e.g. infusion pump) to the venous cannula is a problem. The bubbles float against, and tend to stick to, the inner surface of the tube, with subsequent loss of efficacy and dosing problems. None of the methods and apparatus described in the state of art provides any working solution to how to avoid such flotation and inhomogenities in a tube connecting an injection system and an injection needle, such as a venous cannula.

### SUMMARY OF THE INVENTION

In view of the needs of the art the present invention provides a device and a method of using the device for administration of a substantially homogeneous fluid preparation to a patient. The device of the invention is a tube that prevents particle flotation and sedimentation in the lumen of the tube, when using the tube in transferring a segregating particulate dispersion. In particular the invention provides a tube for use in continuous infusion of an ultrasound contrast agent.

A tube which has a non-circular internal cross-section and which is twisted along its centerline will cause a helical laminar flow pattern that effectively will counteract the effect of gravity and flotation and avoid particle accumulation to the tube walls. The helical motion path of particles around the tube centerline will cause flotation effects to cancel with respect to the particles overall distance from the centerline, and will thus prevent particles from approaching the tube wall. A first aspect of the invention is a tube which is twisted along its centerline and which has a non-circular internal cross-section.

The use of the tube is preferentially in continuous infusion of gas-containing ultrasound contrast agents, as the tube will minimize flotation of the gas microbubbles, enabling administration of a homogeneous preparation.

### BRIEF DESCRIPTION OF THE DRAWINGS

FIG. 1a is a perspective view of a tube according to the invention having an oval compressed internal cross-section.

FIG. 1b is a cross-sectional drawing of the tube of FIG. 1a.

FIGS. 2a and 3a are perspective views of tubes according to the invention having different internal cross-sections comprising 3 rounded lobes.

FIGS. 2b and 3b are cross-sectional drawings of the tubes of FIGS. 2a and 3b, respectively.

FIG. 4 illustrates how a particle in a fluid moves along by the helical flow in a tube of the invention.

FIG. 5 illustrates a concentric tube arrangement.

FIG. 6 illustrates an infusion apparatus including a syringe carrying a contrast agent, an infusion line, an infusion fluid bag, a concentric tube arrangement, a non-circular twisted tube and an intravenous cannula.

### DETAILED DESCRIPTION OF THE INVENTION

Ultrasound contrast agent bubbles that are dispersed in water will float upwards at a rate in the order of a few millimeters per minute. When such bubbles are infused into a patient via a plastic tube that connects the infusion pump to the venous cannula, this flotation will cause an accumulation of bubbles in the upper fluid layers in horizontal sections of the tube. The bubbles might stick to the tube wall and accumulate inside the tube, with a resulting reduction in the dose delivered to the patient. The current invention consists of introducing a helical motion to the bubbles path inside the tube. This is achieved by using a tube having a non-circular internal cross-section, and in addition giving the tube a twisted shape along its length, thus forcing the column of fluid inside the tube to rotate as the fluid is propagated along the tube. A bubble that is transported in a fluid flow system with the above mentioned properties will have a spiral trajectory around the tube centerline. The small velocity component of the bubble caused by flotation will continuously change its direction with respect to the distance vector between the bubble and the tube centerline. The time-averages motion component caused by flotation will for this reason become zero, and no long-term changes in the particles distance from the tube centerline will be expected.

Thus according to one aspect of the present invention there is provided a tube with a non-circular internal cross-section and wherein the tube is twisted along its centerline. The internal cross-section is defined as the cross-section of the lumen extending from the centerline of the tube to the inner wall of the tube. The centerline of the tube is the axis going through the center of the lumen of the tube extending throughout the tube length.

The tube of the invention may be used in transporting any segregating particulate dispersion between two points. By the term segregating is meant that the dispersion may comprise particles that either tend to float or sediment. Using the tube of the invention transportation of segregating particles between two points can be achieved with reduced flotation or sedimentation, minimizing the tendency of the particles to attach to the Inner tube wall. Preferably, the tube is used in administration of an ultrasound contrast agent comprising a dispersion of gas-microbubbles to a patient. The administration may be by bolus injection or by continuous infusion, but is preferably used in infusion procedures as the flotation problem increases over time.

A further aspect of the invention is hence a method of administering a dynamic particulate dispersion to a subject by continuous infusion, wherein the dispersion is delivered from an apparatus comprising a tube as described.

The dimensions of the tube and the velocity of the fluid introduced into the tube are important factors in order to counteract the floating or sedimenting tendency of the particles in the fluid and to achieve a helical laminar flow.

The twisted tube of the invention preferably has an internal cross-section of 2-10 mm². More preferably the cross-section is 4-8 mm², and most preferably the cross-section is about 6 mm², corresponding to an average radius of about 1.5 mm. As the lumen (canal in the tube) is not circular the cross-sectional area is the preferred measure. If the internal volume of the tube is increased further by increasing the cross-sectional area, a problem is that an increased volume of the dispersion to transfer must also be increased.

The shape of the external cross-section of the tube is of limited relevance and could hence be circular or non-circular. A tube with an external circular cross-section is although preferred as this might increase the strength of the tube. Preferably the external (outside) diameter of the tube is 2-6 mm. The outer dimensions of the tube depends on the material chosen for the tube and of course the shape of the internal cross-section.

Typical flow values through a tube of the invention are 100-1000 ml/hr during use, and more preferably 200-500 ml/hr. Typically 500 ml bags of carrier liquid are used in intravenous infusions, and this fluid volume may for instance be administered during a 30-minutes period.

Derived from the preferred flows and cross-section areas given above the average fluid velocity is preferably 0.2-15 cm/s. The velocity of the fluid at the centerline of the tube, which is always higher than towards the walls, is subsequently preferably about 0.4-40 cm/s. The flow should be high in order to counteract the flotation tendency. Typically an ultrasound contrast microbubble float 1-2 mm/min when dispersed in water.

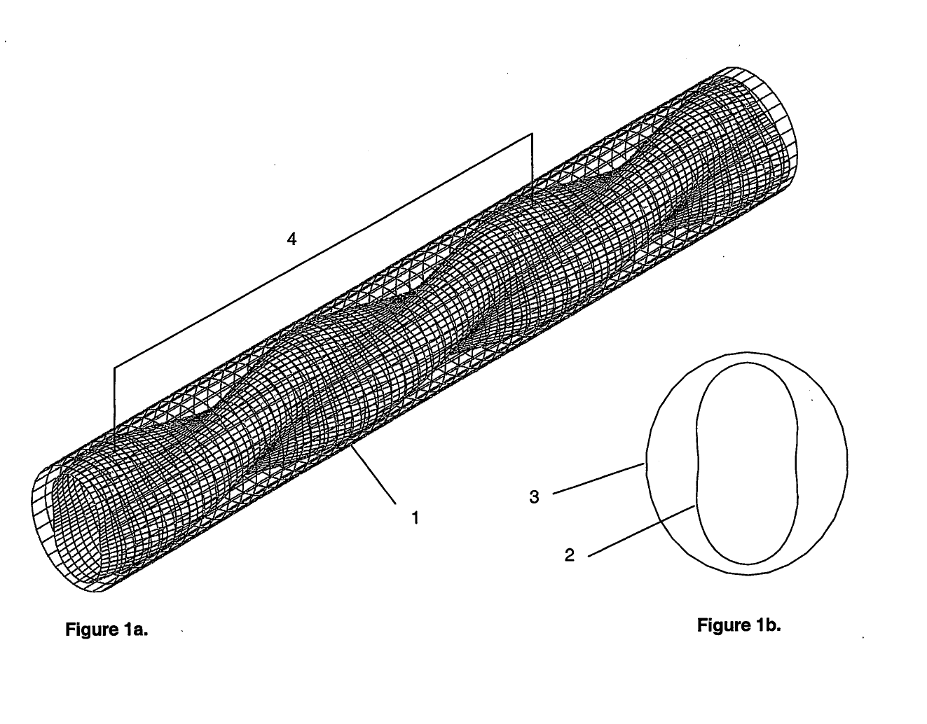

The inner cross-section of the tube is non-circular. Preferably there is a rotational symmetry such that the radius as a function of the angle from the centerline should be a pattern that repeats itself an integer number for each revolution. By the term non-circular is meant that the distance from the center point of a cross-section to the tube wall is varying in at least two directions. The degree of deviation from circular form is preferably at least a 5% difference in distance between the maximum and minimum radius (distance from center to wall). Preferably the tube has a cross-sectional internal geometrical configuration selected from the group consisting of oval, elliptical, triangular, wedge shaped, square, or a configuration comprising 2-5 rounded lobes, and combinations thereof. Particularly preferred shapes are rounded and/or compressed configurations of the above to minimize outgoing sharp edges and corners, and forming of eddies. Most preferred tubes have internal cross-sections formed as a compressed oval configuration or configurations comprising 3 rounded lobes, resembling trefoils, as illustrated in the FIGS. 1-3. FIG. 1a more specifically illustrates a tube 1 according to the invention having a compressed oval inner cross-section. FIG. 1b shows that this tube has a compressed oval inner cross-section 2 and a circular external cross-section 3. The oval configuration is compressed along the shortest axis of the cross section towards the center point of the tube.

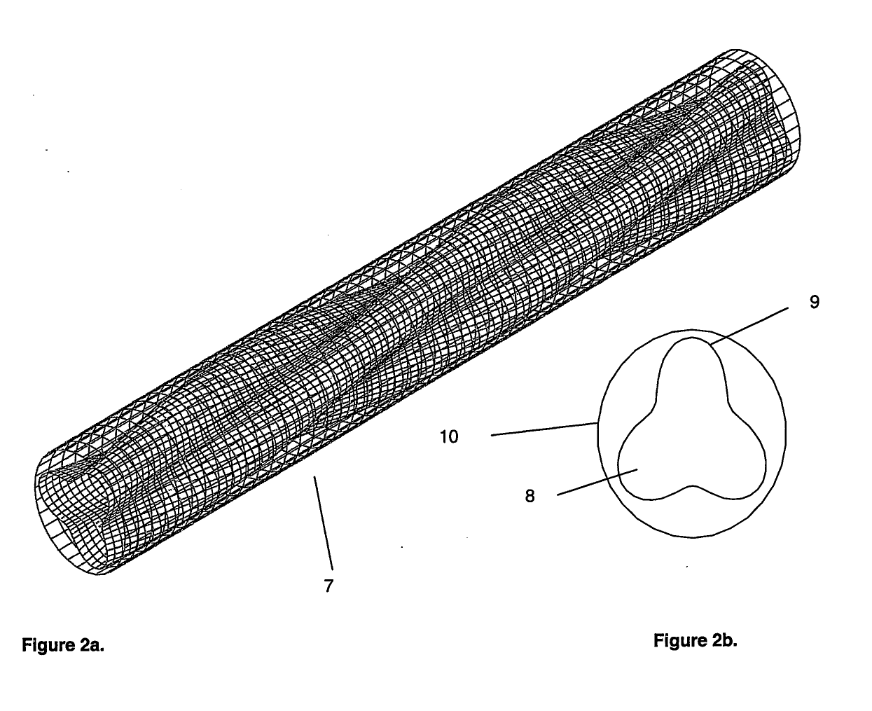

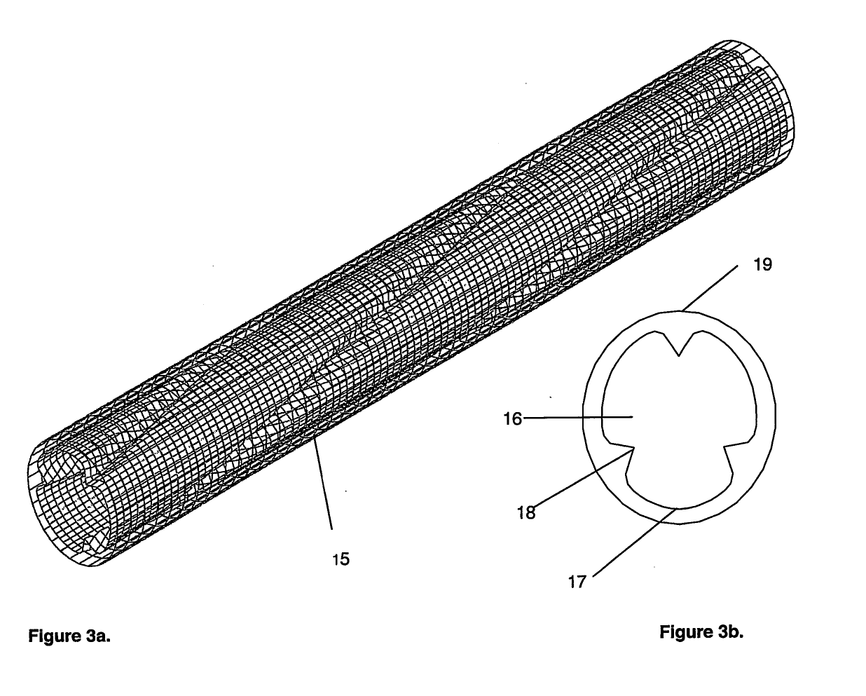

The cross-section of the tube is constant throughout the tube. That is, the shapes of the internal cross-sections are the same throughout the tube. However, although the cross-section is the same along the centerline, the tube is twisted at a constant pitch such that the cross-sections are rotated along the centerline of the tube. Preferably the degree of twisting (pitch) is constant throughout the tube length, such that the distance along the tubing for each revolution of the cross-section is constant. Preferably the pitch is 2 cm-100 cm, and more preferably 5-20 cm and most preferably 7-15 cm. It is important that the pitch of the helical twist along the tube is not too steep, as this might break up the laminar flow pattern. In FIG. 1a the pitch 4 shows the 360° revolution of the internal cross-section of the tubing. FIG. 2a illustrates another tube 7 of the invention having an inner cross-section comprising 3 rounded lobes, symmetrically around the centerline of the tube. FIG. 2b is a cross-sectional view of the tube of FIG. 2a showing the inner cross-section 8 with the rounded lobes 9, and the external circular cross-section 10. FIG. 3a is a third example of a tube 15 of the invention having an inner cross-section comprising 3 lobes. As can be seen from the cross-sectional drawing 3b the inner cross-section 16 also here comprises 3 rounded lobes 17, but a steeper cleft 18 separates these in this tube than in tube 7. The outer cross-section 19 is circular.

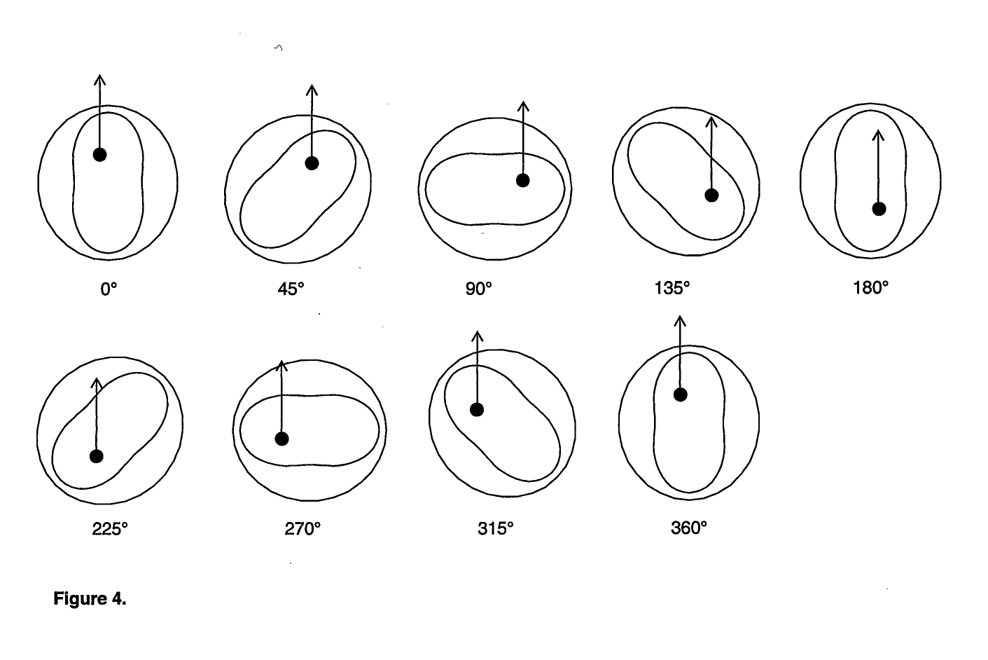

FIG. 4 illustrates how the position of a flotating bubble (black dot) located off-center in a tube of the invention is moved along by the helical flow pattern of the fluid, shown at intervals along the tube corresponding to 45° of rotation of the tube internal cross-section. The direction of upward flotation (arrow) has an angle that Is continuously changing with respect to the bubbles' position relative to the centerline of the tube. The position of a bubble with respect to the centerline will thus remain unchanged during one complete revolution of the column of fluid carrying the bubble.

The tube of the invention is preferably made of a material being soft and flexible, and which at the same time has sufficient strength and limited permeability to gas exchange. Appropriate materials are Fluorplastic, Liquid-Crystal Polymer, Nylon, PEEK, Polycarbonate, Polyimide, Polypropylene, Polyurethane, PTFE, PVC, Silicone, Thermoplastic Elastomere and Polyethylene. Preferred materials are the soft and flexible materials Nylon, Polypropylene, Polyurethane, PVC, Silicone and Thermoplastic Elastomere.

The twisted tube of the invention may be manufactured from a preformed tube by twisting and heating the tube in a separate step, and hence generate the twisted pattern. The preformed tube may have a standard circular inner cross-section or may have a non-circular inner cross-section. The manufacturing process hence comprises modification of a preformed tube by heating and twisting. The preformed non-twisted tube is preferably manufactured in a conventional extrusion process. It is however more preferred to manufacture a tube of the invention by a process wherein both the twisting of the tube and the manufacturing of the non-circular internal cross-section is made in one operation. This can be achieved by an extrusion process, using an extruder comprising a “twisted” nozzle, enabling extrusion of the twisted tube in a continuous process. The nozzle of the extruder should hence have a configuration complying with the internal cross-section of the tube to manufacture, and should also comply with the shape of a short section of the tube. The process of manufacturing the tube comprises the steps of introducing the tube material into the extruder with the twisted nozzle, and setting the nozzle to rotate at a set speed providing the requested degree of twisting to the tube. As the nozzle of the extruder resembles a short section of the tube, it does not have to rotate, i.e. it could be a stationary nozzle, but the manufacturing process would then not be continuous.

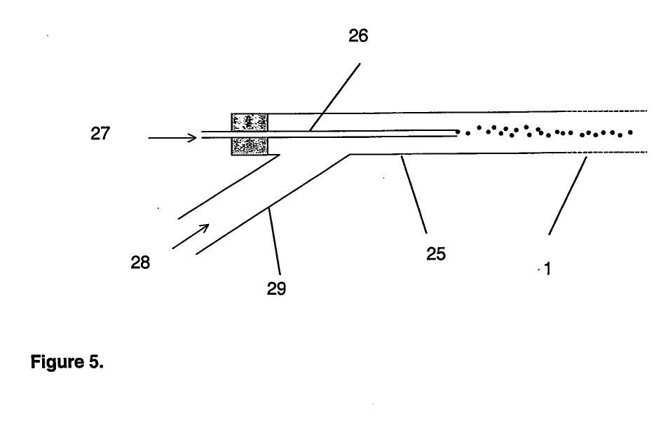

When using the tube in administration of an ultrasound contrast agent further improvements in the system performance can be achieved by introducing the contrast agent bubbles in the center of the tube by using a concentric tube arrangement, for instance as the one shown in FIG. 5. All bubbles will then be carried along the tube without contacting the tube wall, and will remain suspended close to the centerline by the helical motion mechanism mentioned above. Such device may be connected to the tube of the invention and to an injector system. In its simplest solution the device comprises two pipes, one within the other, having the same center. The inner pipe ensures that the contrast agent is injected into the center of the tubing, while the outer pipe is connected to the tube and the injection system. The concentric tube arrangement 25 shown in FIG. 5 may be connected to a delivery device and has an inner pipe 26 with an opening for introducing a dispersion 27. Further a flushing medium 28 may be introduced through the arm 29. As indicated a tube 1 according to the invention may be connected to one end of the concentric tube arrangement.

Hence, a preferred embodiment of the Invention is a method of administering a segregating dispersion by continuous infusion, wherein the dispersion is delivered from an apparatus comprising a tube with a non-circular internal cross-section and wherein the tube is twisted along its centerline, and a concentric tube arrangement connected to one opening of the tube for introduction of the dispersion into the center of the tube.

Co-administration of the dispersion with an admixed flushing medium further enhances product homogeneity, e.g. by reducing the residence time of the dispersion in the tubing, thereby reducing its susceptibility to gravity segregation. Admixture with flushing medium also permits particularly efficient control of administration of the dispersion since the flow rates of both the dispersion and the flushing medium may be independently controlled.

Admixture of the dispersion with flushing medium almost immediately prior to administration to a subject is particularly advantageous in the administration of dispersions such as gas microbubble-containing contrast agents, which may show instability if stored in diluted form, e.g. if diluted prior to transfer into a syringe or other delivery vessel. The use of the tube is preferentially by admixture of an ultrasound contrast agent with an inactive carrier liquid such as saline, Ringer's solution or isotonic glucose. Mixing of the dispersion and flushing medium may, for example, be effected in a three-way connector, e.g. a T-piece, a Y-piece or a tap such as a three way stopcock, or by using a device as illustrated in FIG. 5.

The length of the tube from the point where the carrier liquid and the contrast agent are combined to the venous cannula should despite the improvements offered by this invention be kept as short as possible in order to minimize transit time. Preferably the tube should be no longer than 300 cm, more preferably not more than 200 cm, and most preferably not more than 100 cm. The orientation of the tube (horizontal, vertical) is not critical, but sharp bends and kinks should be avoided, since this might disturb the laminar flow pattern.

The tube and method of the invention may be particularly useful in administration of the ultrasound contrast agents known as Optison®, Sonazoid®, Levovist®, Albunex®, Definity® and Imagent®.

The tube of the invention may be used in apparatus used for administration of a segregating dispersion, replacing conventional tubes having a circular cross-section. Preferably, the tube is combined with known apparatus used in administration of contrast agents, such as gas-containing ultrasound contrast agents, to a patient. A further aspect of the invention is an apparatus for use in administration of a gravity segregating dispersion to a subject, said apparatus comprising a tube with a non-circular internal cross-section and which is twisted along its length. Preferably, the apparatus further comprises:
i) a delivery device adapted to receive and deliver a dispersion,
ii) an intravenous cannula.

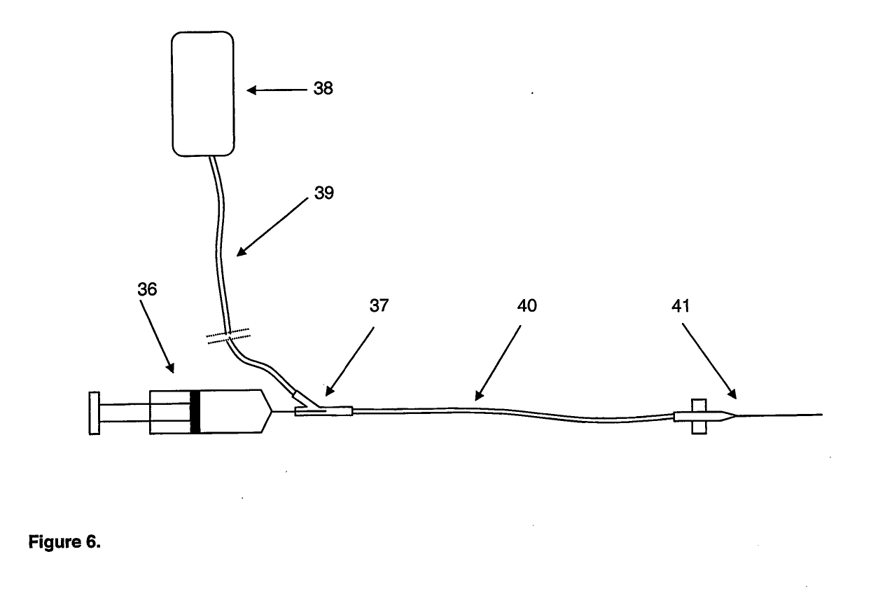

The delivery device may for instance be an injection system comprising an infusion pump adapted to expel the dispersion at a given rate, and a delivery receptacle. The delivery receptacle may be a syringe or cartridge or a flexible bag container. Such apparatus may further include a concentric tube arrangement to ensure introduction of the dispersion from the delivery device to the center of the tube of the invention. Even further, the apparatus may include mixing means adapted to admixture the dispersion with a flushing medium. FIG. 6 shows an apparatus for administration of a contrast agent by continuous infusion. The apparatus includes a syringe 36 carrying a contrast agent. The syringe is preferably placed in an infusion pump for controlled expel of the contrast agent. The syringe nozzle/needle is connected to a concentric tube arrangement 37 as also shown in FIG. 5. Admixture of the contrast agent with a flushing medium is enabled by connecting an infusion fluid bag 38 to an infusion line 39 for transport of the flushing medium to the concentric tube arrangement 37 where the mixing is effected. The concentric tube arrangement 37 ensures that the contrast agent is introduced into the center of one opening of the non-circular twisted tube 40. The second end of the tube 40 is connected to an intravenous cannula 41 for injection of the diluted contrast agent to a patient.

An infusion apparatus as described above, including a tube of the invention, may also comprise additional means for maintaining a segregating dispersion homogeneous during infusion. Such means may comprise devices for subjecting the dispersion to different agitation mechanisms.

Use of the tube described, and apparatus comprising such tube, in administration of a segregating dispersion, such as an ultrasound contrast agent, by continuous infusion is yet another aspect of the invention. The tube may further be used as an intravenous catheter. While the preferred embodiment of the present invention has been shown and described, it will be obvious in the art that changes and modifications may be made without departing from the teachings of the invention. The matter set forth in the foregoing description and accompanying drawings is offered by way of illustration only and not as a limitation. The actual scope of the invention is intended to be defined in the following claims when viewed in their proper perspective based on the prior art. The following non-limitative examples serve to illustrate the invention.

### EXAMPLE

### Example 1

A tube (40) with an elliptical inner cross section according to FIG. 1a is used. The inner smallest diameter is 3 mm, and the largest diameter is 6 mm. The distance along the tube for a full revolution of the helix is 5 cm. A two meter piece of this tube is connected as shown in FIG. 5 and FIG. 6. The tube is placed horizontally along a straight line. A syringe (36) is filled with Optison®, and is mounted in an infusion pump that delivers a flow of 1 mL/min. An infusion bag (38) is filled with isotonic glucose solution, and the flow from this bag is adjusted to 8 mL/min. The content of the tube (40) is inspected, and the white streak of fluid containing bubbles (Optison) is observed to move along the centre of the tube with a velocity of about 2 cm/s without contacting the tube wall.

### Example 2 (Comparative)

The experiment described in example 1 is repeated, but with a tube with a circular inner cross section with an inner diameter of 4.2 mm. The tube has the same length and about the same internal cross-sectional area as the tube used in example 1. It is observed that the white streak of fluid that contains bubbles slows down and deviates upwards from the centreline towards the distal end of the tube. Also, a layer of bubbles sticking to the upper, inner surface of the tube is observed to build up towards the end of the tube.

What is claimed is:

1-15. (canceled)

16. Contrast agent administration apparatus comprising a tube with a non-circular internal cross-section, wherein the tube is twisted along the centerline of the tube.

17. An apparatus as claimed in claim 1, wherein the tube has an internal cross-section of 2-10 mm².

18. An apparatus as claimed in claim 1, wherein the tube has an external circular cross-section.

19. An apparatus as claimed in claim 1, wherein the tube has an oval cross-section.

20. An apparatus as claimed in claim 1, wherein the tube has a cross-section comprising 2-5 rounded lobes.

21. An apparatus as claimed in claim 1, wherein the tube is twisted at a constant pitch.

22. An apparatus as claimed in claim 1, wherein the tube is made of a material selected from Fluorplastic, Liquid-Crystal Polymer, Nylon, PEEK, Polycarbonate, Polyimide, Polypropylene, Polyurethane, PTFE, PVC, Silicone, Thermoplastic Elastomere and Polyethylene.

23. An apparatus as claimed in claim in 1 further comprising a concentric tube arrangement connected to one opening of the tube for introduction of a contrast agent into the centre of the tube.

24. Apparatus as claimed in claim 1 further comprising:
i) a delivery device adapted to receive and deliver a dispersion,
ii) an intravenous cannula.

25. An apparatus as claimed in claim 8 further comprising a concentric tube arrangement connected to one opening of the tube and to the delivery device for introduction of a contrast agent into the centre of the tube, and wherein a second end of the tube is connected to the intravenous cannula.

26. A method of manufacturing a tube with a non-circular internal cross-section, wherein the tube is twisted along the centerline of the tube, by continuous extrusion by the following steps:
i) introducing the tube material into a extruder comprising a nozzle having a configuration complying with the internal cross-section and a short section of the tube to be manufacturing,
ii) setting the nozzle to rotate at a set speed.

27. A method of administering a contrast agent to a subject using a tube with a non-circular internal cross-section, wherein the tube is twisted along the centerline of the tube.

28. A method as claimed in claim 12 wherein the contrast agent is an ultrasound contrast agent comprising gas microbubbles and wherein the administration is by continuous infusion.

29. A method as claimed in claims 10 wherein the contrast agent is admixed with a flushing medium prior to administration to the subject.

30. A method of administering a contrast agent to a subject using an apparatus as claimed in claim 8.

31. A method of administering a contrast agent to a subject using an apparatus as claimed in claim 9.

32. A method of administering a contrast agent to a subject using an apparatus as claimed in claim 10.
